(19) United States
(12) Patent Application Publication
(10) Pub. No.: US 2018/0333293 A1
(43) Pub. Date: Nov. 22, 2018

(54) VEHICLE SEAT WITH WARMING AND COOLING EFFECT FOR SPORTS ACTIVITIES

(71) Applicant: Ford Global Technologies, LLC, Dearborn, MI (US)

(72) Inventors: Jeroen Lem, Maastricht (NL); Philipp Wolf, Cologne (DE); Nikica Hennes, Aachen (DE)

(73) Assignee: Ford Global Technologies, LLC, Dearborn, MI (US)

(21) Appl. No.: 15/925,895

(22) Filed: Mar. 20, 2018

(30) Foreign Application Priority Data
May 18, 2017 (DE) 102017208387.5

(51) Int. Cl. A61F 7/00; B60N 2/56; B60N 2/90; A61H 7/00

(52) U.S. Cl. CPC A61F 7/00; B60N 2/5685; B60N 2/976; A61H 7/00; A61H 2203/0431; A61H 2201/0214; A61H 2201/10; A61H 2201/0149; A61F 2007/0093; A61H 2201/0207

(57) ABSTRACT

An arrangement of a vehicle seat with a control device and an input device is made available, in the seat surface and backrest of which devices are integrated for applying a forceful contact or a climatic effect. The arrangement is suitable in particular for warming a person up before a sports activity or for cooling a person down after a sports activity.

### FIELD OF THE INVENTION

The present invention generally relates to vehicle seating, and more particularly relates to an arrangement of a vehicle seat with devices for applying forceful contacts and climatic effects in accordance with a sports activity of a person sitting on the vehicle seat.

### BACKGROUND OF THE INVENTION

People participating in sports typically warm up before a sports activity and also often cool down after a corresponding activity. However, if there is insufficient time, these exercises in corresponding training units are often too short. There is the added fact that people taking part in sports often travel to the location of the sports activity by motor vehicle, in particular by a car. The sports activity is therefore often performed with the cold muscles, which poses an increased threat of injuries. It would be desirable to provide a vehicle seat that can afford car drivers the possibility of effective warming up before a sports activity and of effective cooling down after a sports activity.

### SUMMARY OF THE INVENTION

According to one aspect of the present invention, a vehicle seat arrangement is provided. The vehicle seat arrangement includes a vehicle seat with a seat surface and a backrest, and at least one device for applying at least one forceful contact and at least one device for applying a climatic effect comprising a temperature and moisture effect to body regions of a person sitting on the seat. The vehicle seat arrangement also includes a control device designed to control application of a forceful contact or the climatic effect in dependence on a sports activity of the person sitting on the seat, and an input device for inputting instructions to the control device.

According to another aspect of the present invention, a seat arrangement is provided. The seat arrangement includes a seat having a seat surface and a backrest, a first device for applying forceful contact and a second device for applying a climatic effect comprising a temperature and moisture effect to body regions of a person sitting on the seat, and a control device controlling application of the forceful contact or the climatic effect based on a sports activity.

According to a further aspect of the present invention, a method for warming up a person before a sports activity and/or cooling down a person after a sports activity with a seat arrangement is provided. The method includes the steps of making available the seat arrangement, inputting a selected person-specific mode relating to intensity of a forceful contact and climatic effect into an input device, starting operation of a device for applying the forceful contact and of a device for applying a climatic effect, and adapting the operation of the devices to specific requirements by manual input of person-specific data and/or automatic reading-in of person-specific data, which data are transmitted by measurement devices to which the person is connected.

These and other aspects, objects, and features of the present invention will be understood and appreciated by those skilled in the art upon studying the following specification, claims, and appended drawings.

### BRIEF DESCRIPTION OF THE DRAWINGS

In the drawings:

FIG. 1 is a schematic view of a vehicle seat employing a seat arrangement, according to one embodiment; and

FIG. 2 is a flow chart of a method of warming up and cooling down a person on the seat, according to one embodiment.

### DETAILED DESCRIPTION OF THE PREFERRED EMBODIMENTS

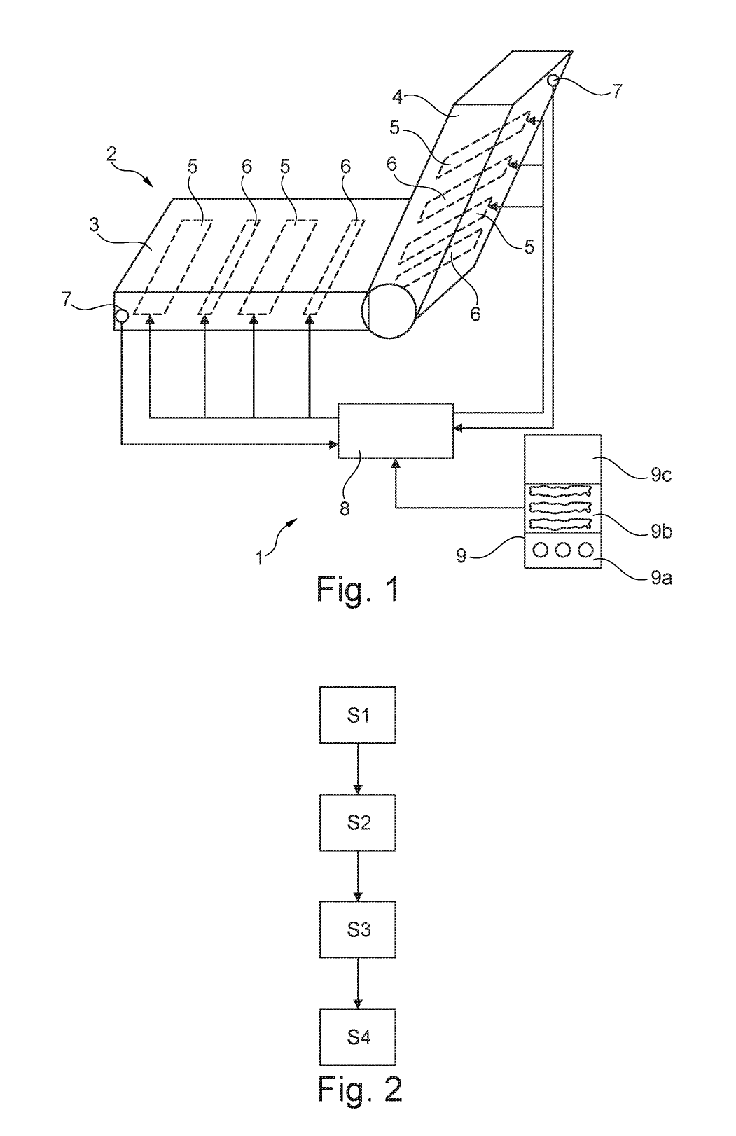

A seat arrangement 1 of a vehicle seat according to one embodiment is shown in FIG. 1. The seat arrangement comprises a seat 2. The seat 2 may use a conventional vehicle seat, in particular of a motor vehicle, and has a seat surface 3 and a backrest 4. The material of the seat 2 comprises cloth and/or leather, for example.

For example, two devices 5 for applying a forceful contact, in short massage devices 5, are arranged in the material of the seat surface 3 and of the backrest 4, respectively. It is also possible for more than two massage devices 5 to be present in the seat surface 3 and the backrest 4, respectively, for example three, four, five or a greater number thereof, in order to ensure an effective massage of a person sitting on the seat 2. The massage devices 5 can be operated pneumatically, e.g., by gas or a liquid, or mechanically, or by a combination of both.

For example, two devices 6 for applying a climatic effect are arranged in the material of the seat surface 3 and of the backrest 4, respectively. The devices 3 may include devices for applying a temperature effect, e.g., a heating mat, and for applying a moisture effect, e.g., a ventilation component which is suitable for removing moisture. A climate control system (CCS) for fine-precision control of a desired temperature can also be used for example, wherein air actively heated or cooled by use of Peltier elements can be directed toward persons. It is also possible for more than two corresponding devices 6 to be present in seat surface 3 and backrest 4, respectively, for example three, four, five or a greater number thereof, in order to ensure an effective application of heat or cooling of body regions of a person sitting on the seat 2. The devices 6 can be designed, among other things, to apply both a heating temperature that is higher than the body temperature of the person and also a cooling temperature that is lower than the body temperature of the person. However, the devices 6 can also be functionally different. For example, some devices can be designed only to apply a higher temperature, others only to apply a lower temperature, and others to apply a moisture effect. In other words, the devices 6 for controlling climatic effects can be arranged in different and mutually independent zones.

Moreover, various sensors 7 including pulse sensor temperature sensor, body moisture sensor and weight sensor, for example, are arranged in the seat 2 for the purpose of determining person-specific measurement data, e.g., pulse, temperature, body moisture and weight of a person. The sensors 7 are connected to a control device 8 to which the measurement data are transmitted. Sensors for determining person-specific measurement data can also be arranged in other devices of the vehicle in which the seat 2 is located. For example, pulse sensors can be arranged in the steering wheel such that a person's pulse can be determined.

The massage devices 5 and the devices 6 for applying a climatic effect are likewise connected to the control device 8, which controls the function of the devices 5 and 6. The control device 8 may be control circuitry such as a microprocessor that is designed to control the devices 5 and 6 according to instructions that it receives from an input device 9 or multiple input devices. The signal paths of the control instructions between input device 9, control device 8 and the devices 5 and 6 and sensors 7 are indicated by the directions of the arrows.

The input device 9 is designed for the input of commands. For this purpose, the input device 9 has various switches 9a and/or buttons for switching on and off and for activating or deactivating defined settings. Moreover, the input device 9 has a keyboard 9b via which written instructions can be given. The instructions are visible on the display 9c of the input device 9. The instructions can be given, for example, as responses to a questionnaire that is shown on the display 9c. The display 9c can also be designed as a touch screen, in which case the entire operation of the input device 9 can take place via the display 9c, and it is then also possible to omit the switches 9a and the keyboard 9b. Moreover, the input device 9 can have a microphone and voice or speech recognition, such that spoken instructions can also be given. For this purpose, a voice control is integrated in the control device 8.

The use of the seat arrangement 1 shown in FIG. 1 can be illustrated by a method of warming up and/or cooling down a seated person before and/or after a sports activity is shown in FIG. 2, according to one embodiment. In a first step S1 of the method, an arrangement 1 as shown in FIG. 1 is made available. In a second step S2, a person-specific mode relating to the intensity of the forceful contacts applied by the massage device 5 and to the level of the temperature to be achieved by the device for applying a climatic effect is input into the input device. This can be done, for example, by input via the keyboard. For this purpose, it is possible, for example, to select a defined person-specific profile which has already been created and stored in a memory of the input device 9 or of the control device 8. For this purpose, various users can each have a personalized profile. However, a specific user can also have different profiles. It is also possible, however, for a defined intensity of the contacts and level of the temperature to be programmed in directly again.

For this purpose, for example, a form can be shown on the display 9c or output audibly, with input fields or questions that are to be suitably completed or with questions and associated boxes that are to be activated by marking with a tick. The input fields or questions can be, for example:
identity of the user?
profile of the user?
time (before the sports activity/after the sports activity)?
nature of the sports activity (type of sport)?
main focus on defined body regions or muscle groups?
existing injury?

In other words, in the second step S2 a predefined procedure relating to the warming or cooling before or after a sports activity is selected or a defined desired procedure is manually programmed. Then, in a third step S3, the operation of the devices 5 and 6 is started.

In a fourth step D4, the activity of the devices 5 and 6 is adapted, during their operation, to specific requirements in the context of warm-up in preparation for training or in the context of cooling down after a sports activity. For this purpose, the function of the devices 5 and 6 can be monitored manually by use of the input device(s). Alternatively or in addition, the function of the devices 5 and 6 can also be adapted to current requirements on the basis of automatically read-in person-specific data, e.g., pulse or temperature, which are transmitted to the control device 8 for example by the sensors 7 to which the person is connected. Thus, for example, the setting or settings can be such that defined body regions are intended to be warmed up and other body regions are intended to be cooled. For this purpose, the devices for applying a climatic effect are controlled accordingly.

In a further embodiment of the method, other components of the vehicle can be included in the warming up before a sports activity and in the cooling down after a sports activity. For this purpose, for example, the steering wheel or the armrests of the vehicle can be heated or cooled.

The present disclosure provides a seat arrangement comprising at least one vehicle seat with a seat surface and a backrest which each have at least one device for applying at least one forceful contact and at least one device for applying a climatic effect to the body regions of a person sitting on the seat. Moreover, the arrangement comprises a control device which is designed to control the application of the forceful contact or of the climatic effect in dependence on a sports activity of the person sitting on the seat, and an input device for the input of instructions, wherein the input device is connected to the control device.

A forceful contact is to be understood as meaning that a force acting on a body region of a person is sensed by the person as pressure. Ideally, it is not just one contact that is exerted but a multiplicity of contacts, such that a massage can be performed. The device for applying at least one forceful contact is therefore also designated as a massage device. The massage device can be operated pneumatically or mechanically, or by a combination of both.

The term climatic effect is to be understood as meaning temperature and moisture effects that can be transferred to the body. Temperature effect is to be understood as meaning that a quantity of thermal energy is transferred to the body that is either higher or lower than the body temperature of the person in question. Moisture effects concern the removal of moisture from the skin for the purposes of drying and/or cooling.

The term cooling down, also referred to as cool-down, relates to a gradual and controlled decrease of physical exertion after a sports activity. Among other things, this loosens the muscles, accelerates the breakdown of metabolic products and returns pulse, blood pressure, breathing and body temperature to normal.

Vehicle seats conventionally have a seat surface and a backrest. The term seat surface is used here for the part of the seat on which a person sits, although more than just the actual upper surface is meant.

The vehicle seat according to one embodiment is advantageous since a mixture of massage and of temperature application can get a person ready for a sports activity, specifically during a car journey. This saves time for the actual warm up and is also advantageous from the point of view of economy since, in the case of a training unit that has to be paid for, the costs are reduced by the shortened warm-up time.

In the seat arrangement according to one embodiment, the control device can preferably control the contact or the climatic effect in temporal dependence on the sports activity. Here, temporal dependence means a relationship to a taking-place before or after a sports activity and moreover also the time to the start of a sports activity or after the end of a sports activity. This is advantageous since the contacts and the climatic effects before a sports activity can be controlled differently than those after a sports activity. For example, the muscles are gradually warmed up before a sports activity and gradually cooled down after a sports activity.

Preferably, in the seat arrangement according to one embodiment, commands relating to the forceful contacts or to the climatic effect can be input manually to the control device by a user of the input device. Advantageously, the user (person) inputs whether warming up or cooling down is required and/or the force with which the contacts are to be applied and the extent to which the climatic effects are to be applied. Particularly, person-specific modes relating to the intensity of the forceful contact(s) and/or of the climatic effect can be input via the input device. In other words, a defined person can be assigned a defined profile in respect of the intensity of the forceful contact(s) and/or the level of the temperature and also the duration, and also a defined body region to be treated, it being possible for the profile to be selected via the input device. Several corresponding profiles can also be present for one person. The one or more profiles can also be assigned to a defined type of sport.

Moreover, in the seat arrangement according to one embodiment, person-specific parameters can preferably be automatically recorded via connections of the control device to recording devices. The parameters may include the body temperature, pulse, blood pressure and other person-specific parameters. The recording devices may be, for example, portable measuring appliances (fitness trackers in the form of watches or chest straps), sensors integrated in the steering wheel, or sensors integrated in the seat according to various embodiments. The recorded parameters are advantageously included in a calculation of an effective warming and/or cooling of the seated person by the devices of the seat. The specific sports activity can also be input, such that the arrangement can advantageously focus on the warming and/or cooling of defined muscle groups.

Moreover, in the seat arrangement according to one embodiment, the control device is connected to a navigation appliance of the vehicle. In this way, the time of arrival at the location of the sports activity can advantageously be determined and the duration, for example, of the warming and the degree of intensity of the effect of the devices of the seat can be adapted to the time.

Moreover, it is preferable if the device for applying a forceful contact and at least one device for applying a climatic effect can be controlled in such a way that a treatment of a defined sports injury is possible. The effect of the devices of the seat can be directed, e.g., manually via the input device, to a defined body region, or a defined profile of the effect of the devices can also be assigned to a defined injury and called up when required.

In the vehicle seat of the seat arrangement according to one embodiment, in order to apply a warming effect and to apply a cooling effect, different devices for applying a climatic effect are present. In this way, defined devices can be designed particularly for warming, i.e., for generating a temperature higher than the body temperature, and other devices can be designed in particular for cooling, i.e., for generating a temperature lower than the body temperature. Advantageously in this way, less energy is needed when changing from warming to cooling. Moreover, defined devices can advantageously be used for warming units and others for cooling.

In the vehicle seat of the seat arrangement according to one embodiment, in order to apply a warming effect before a sports activity and to apply a cooling effect after a sports activity, different devices for applying forceful contact are present. The disclosure also relates to a motor vehicle having the seat arrangement. The disclosure further relates to a method for warming a person up before a sports activity and/or cooling a person down after a sports activity by use of the seat arrangement, with the following steps:
making available the arrangement;
inputting a selected person-specific mode relating to the intensity of the forceful contacts and climatic effect into the input device,
starting the operation of the device for applying at least one forceful contact and of the device for applying a climatic effect; and
adapting the operation of the devices to specific requirements by manual input of person-specific data and/or automatic reading-in of person-specific data, which data are transmitted by measurement devices to which the person is connected.

It is to be understood that variations and modifications can be made on the aforementioned structure without departing from the concepts of the present invention, and further it is to be understood that such concepts are intended to be covered by the following claims unless these claims by their language expressly state otherwise.

What is claimed is:

1. A vehicle seat arrangement comprising:
a vehicle seat with a seat surface and a backrest;
at least one device for applying at least one forceful contact and at least one device for applying a climatic effect comprising a temperature and moisture effect to body regions of a person sitting on the seat;
a control device designed to control application of a forceful contact or the climatic effect in dependence on a sports activity of the person sitting on the seat; and
an input device for inputting instructions to the control device.

2. The seat arrangement as claimed in claim 1, wherein the control device controls the contact or the climatic effect in temporal dependence on the sports activity.

3. The seat arrangement as claimed in claim 1, wherein by use of the input device, commands relating to the forceful contact or to the climatic effect can be input manually to the control device.

4. The seat arrangement as claimed in claim 3, wherein person-specific modes relating to intensity of the forceful contact and/or of the climatic effect can be input via the input device.

5. The seat arrangement as claimed in claim 1, wherein person-specific parameters can be automatically recorded via connections of the control device to one or more recording devices.

6. The seat arrangement as claimed in claim 1, wherein the control device is connected to a navigation appliance of the vehicle.

7. The seat arrangement as claimed in claim 1, wherein the device for applying at least one forceful contact and the at least one device for applying a climatic effect controlled to treat a defined sports injury is possible.

8. The seat arrangement as claimed in claim 1, wherein in order to apply a warming effect before a sports activity and to apply a cooling effect after a sports activity, different devices for applying a climatic effect are present.

9. The seat arrangement as claimed in claim 1, wherein in order to apply a warming effect before a sports activity and to apply a cooling effect after a sports activity, different devices for applying a forceful contact are present.

10. A seat arrangement comprising:
a seat having a seat surface and a backrest;
a first device for applying forceful contact and a second device for applying a climatic effect comprising a temperature and moisture effect to body regions of a person sitting on the seat; and
a control device controlling application of the forceful contact or the climatic effect based on a sports activity.

11. The seat arrangement as claimed in claim 10, wherein the control device is configured to control the contact or the climatic effect in temporal dependence on the sports activity.

12. The seat arrangement as claimed in claim 10 further comprising an input device connected to the control device for inputting instructions, wherein by use of the input device, commands relating to the forceful contact or to the climatic effect can be input manually to the control device.

13. The seat arrangement as claimed in claim 12, wherein person-specific modes relating to the intensity of the forceful contacts and/or of the climatic effect can be input via the input device.

14. The seat arrangement as claimed in claim 10, wherein person-specific parameters can be automatically recorded via connections of the control device to one or more recording devices.

15. The seat arrangement as claimed in claim 10, wherein the control device is connected to a navigation appliance of the vehicle.

16. The seat arrangement as claimed in claim 10, wherein the device for applying at least one forceful contact and at least one device for applying a climatic effect are controlled to treat a defined sports injury.

17. The seat arrangement as claimed in claim 10, wherein in order to apply a warming effect before a sports activity and to apply a cooling effect after a sports activity, different second devices for applying a climatic effect are present.

18. The seat arrangement as claimed in claim 10, wherein in order to apply a warming effect before a sports activity and to apply a cooling effect after a sports activity, different first devices for applying a forceful contact are present.

19. The seat arrangement as claimed in claim 10, wherein the seat is located on a motor vehicle.

20. A method for warming up a person before a sports activity and/or cooling down a person after a sports activity with a seat arrangement, comprising:
making available the seat arrangement;
inputting a selected person-specific mode relating to intensity of a forceful contact and climatic effect into an input device;
starting operation of a device for applying the forceful contact and of a device for applying a climatic effect; and
adapting the operation of the devices to specific requirements by manual input of person-specific data and/or automatic reading-in of person-specific data, which data are transmitted by measurement devices to which the person is connected.
